(19) United States
(12) Patent Application Publication
(10) Pub. No.: US 2008/0171790 A1
(43) Pub. Date: Jul. 17, 2008

(54) Fatty acids-systemic lipid solubilizers conjugates

(76) Inventors: Filiberto Palmiro Zadini, North Hills, CA (US); Giorgio Cesare Zadini, Camarillo, CA (US)

Correspondence Address: GIORGIO ZADINI, 2237 HILLTOP LANE, CAMARILLO, CA, 93012, US

(21) Appl. No.: 12/008,474

(22) Filed: Jan. 10, 2008

Related U.S. Application Data
(60) Provisional application No. 60/880,075, filed on Jan. 12, 2007.

(51) Int. Cl. A61K 31/20

(52) U.S. Cl. 514/558

(57) ABSTRACT

A pharmacological compound characterized by the conjugation of a fatty acid such as butyric, caproic, caprylic, lauric, myristic, palmitic, stearic, arachidic, behenic, oleic, linoleic, alpha-linolenic, arachidonic, eicosapentaenoic, docosahexaenoic acid, euric acid with Hyodeoxycholic acid or other systemic biocompatible lipid solubilizer/detergent compounds, as opposed to biocompatible lipid solubilizers/detergents sequestered in the entero-hepatic circulation, said conjugated compound being capable of producing a sustained bioavailability in the systemic circulation to access atherosclerotic plaques and cause ubiquitous dissolution of the cholesterol crystals and of the other cholesterol/lipids aggregations forming the lipidic component of an atherosclerotic plaque and also the ubiquitous dissolution of adipose tissue.

### RELATED CASES

This Application corresponds to Applicants Provisional Patent Application No. 60/880,075 filed on Jan. 12, 2007 entitled “Fatty Acids—Systemic Lipid Solubilizers Conjugates”.

### FIELD OF THE INVENTION

This application consists of a delipidizing pharmacological compound useful in treatment of atherosclerosis and obesity.

### BACKGROUND OF THE INVENTION

The invention consists of compounds characterized by the conjugation of a certain fatty acid with a lipid solubilizer/detergent capable of producing a sustained bioavailability in the systemic circulation to access atherosclerotic plaques and cause dissolution of the cholesterol crystals and of the other cholesterol/lipids aggregations forming the lipidic component of an atherosclerotic plaque.

Gilat & al., working on pharmacological compounds aimed at dissolving gallstones, have noted that compounds resulting from conjugation of certain fatty acids with cholic acid via an amid bond are capable of dissolving cholesterol crystals at much higher rate that plain biliary acids in the bile environment. By considering that bile acids are not the major solubilizers of cholesterol in bile when cholesterol is in a crystallized state of aggregation, such as in the gallstones, particularly in the usually supersaturated human bile and by considering that phospholipids were rather shown to be the major physiologic cholesterol solubilizers in bile, able to normalize the rapid cholesterol crystallization, the authors first investigated whether parts of the phospholipid molecule such as fatty acids and also mono- or diglycerides could act as cholesterol solubilizers. Their studies revealed that saturated long chain fatty acids and some other parts of the phospholipid molecule had cholesterol solubilizing activity on cholesterol crystals. The FABACs (Fatty Acid Bile Acid Conjugates) were conceived and designed with the purpose of delivering a cholesterol solubilizing molecule (a long chain fatty acid) into bile using the very efficient entero-hepatic circulation of bile salts. As the Authors report in their abstract, the rationale was to combine a cholesterol solubilizing moiety (a saturated fatty acid) with a bile acid (cholic acid) as a vehicle to enable secretion into bile and entry into the entero-hepatic circulation.

The Authors report that in vitro and animal studies confirmed that FABACs are both able to prevent biliary cholesterol crystallization and dissolve preformed cholesterol crystals in rodents fed a lithogenic diet. In gallstone susceptible mice, FABACs prevented gallstone formation and dissolved gallstones. The Authors add that FABACs were found to be able to decrease blood cholesterol, and, referring to article Fatty acid bile acid conjugates inhibit atherosclerosis in the C57BL/6 mouse model, by Gonen A, Shaish A, Leikin-Frenkel A, Gilat T, Harats D, the Authors report beneficial effect on atherosclerosis, meaning beneficial effect on atherosclerosis progression and blood lipid levels in mice, as the referred article discloses.

### SPECIFICATIONS

With the present invention Applicants propose the conjugation of a fatty acid such as for instance butyric, caproic, caprylic, lauric, myristic, palmitic, stearic, arachidic, behenic, oleic, linoleic, alpha-linolenic, arachidonic, eicosapentaenoic, docosahexaenoic acid, euric acid with a biocompatible lipid solubilizer/detergent, the detergent having the property not only of functioning as a vehicle capable of maintaining a sustained bioavailability of the Fatty Acid in the systemic circulation, rather than in the entero-hepatic circulation as the FABACs disclosed by Gilat, but also functioning as a solubilizer/detergent/surfactant/emulsifier of cholesterol, the compound being capable of accessing the cholesterol deposits of the atherosclerotic plaques or via crossing of the fibrous cap or via the vasa vasorum.

A model of a biocompatible detergent having the disclosed properties of escaping in significant proportion the enterohepatic circulation and of avoiding therefore of being trapped and sequestered in the enterohepatic circulation is the Hyodeoxycholic acid. Hyodeoxycholic acid/salt has among the all known biliary acids/salts the unique property of indeed being excreted in the urine in large amount after oral administration. Analysis of urinary bile acids showed that a large proportion, up to 84%, of the administered hyodeoxycholic acid is excreted in the urine as a glucuronide as opposite to any other known biliary acid salts of which the amount excreted in the urine is negligible to zero. The bilary acids/salts excluding the hyodeoxycholic acid typically escape the systemic circulation and are sequestered in the enterohepatic circulation making themselves very poor candidates as active medications to be used in regression of atherosclerotic plaques through a mechanism of direct solubilization of the plaque lipidic core, unless a modification in their molecule allows them to leave and escape the enterohepatic circulation and be bioavailable for a reasonably sustained period of time in the systemic circulation. Besides the hyodeoxycholic acid, there are other biological biocompatible lipid solubilizers/emulsifiers that share with the hyodeoxycholic acid the properties of not entering the entero-hepatic circulation and of becoming available, therefore, in the systemic circulation where they can exert their beneficial effects. Gilat's FABACs use the biliary acid as vehicle to transport the Fatty acid to target i.e. to attack cholesterol gallstones. He does not mention nor he hints to the conjugation of fatty acids with systemic lipid solubilizers that offer the above disclosed advantages. It is expected that a systemic lipid solubilizer reinforced with conjugation with any of the above listed Fatty acids will act upon lipid/cholesterol aggregates causing regression of atherosclerotic plaques. It is expected that the crystallized cholesterol aggregations in the cholesterol plaques could be a particularly vulnerable target for such compounds. Another target could be the adipose tissue, with probable dissolution of adipose tissue, particularly for those compounds capable of reaching the subcutaneous tissue. All the above are therapeutic events impossible to achieve by Gilat's compounds, as Gilat's compounds are sequestered in the entero-hepatic circulation.

The systemic biological/biocompatible lipid solubilizers that can be conjugated with fatty acids can be classified according to several criteria.

### Lipid Solubilizers Classified According to Structure:

Alkyl glycosides, which include: -D-glucopyranoside, □-D-glucopyranoside, n-octyl-□n-nonyl-D-glucopyranoside, □-D-glucopyranoside, n-hexyl-□n-heptyl-Dthioglucopyranoside, and □-D-maltoside, octyl-□-D-maltoside, decyl-□dodecyl-maltoside and others.

Glucamides, which include: MEGA-10, MEGA-9, MEGA-8, Deoxy Big CHAP, CHAP, and others.

Polyoxyethylenes, monodisperse and polydisperse which include: reduced TRITON® X-100, reduced TRITON® X-114, TRITON® X-100, NP-40, TRITON® X-114, GENAPOL® X-080, GENAPOL® X-100, C12E8, C12E9, THESIT®, LUBROL® PX, GENAPOL® C-100, BRIJ® 35, PLURONIC® F-127®, (laurate), TWEEN® 20 (oleate), TWEEN® 80, and others.

Zwittergents, which include: EMPIGEN BB® (n-dodecyl-N,Ndimethylglycine), ZWITTERGENT® 3-08, ZWITTERGENT® 3-10, ZWITTERGENT® 3-12, ZWITTERGENT® 3-14, ZWITTERGENT® 3-16, CHAPS, CHAPSO, and others.

### Lipid Solubilizers Classified According to Electric Charges:

Ionic Lipid solubilizers/detergents, which include:

### BATC

### Cetyltrimethylammonium Bromide (CTAB), Molecular Biology Grade

### Lauroylsarcosine, Sodium Salt

Sodium n-Dodecyl Sulfate (SDS)
Sodium n-Dodecyl Sulfate (SDS), High Purity
Sodium n-Dodecyl Sulfate (SDS), Molecular Biology Grade
Sodium n-Dodecyl Sulfate (SDS), 30% Solution

### TOPPS

### Non-Ionic Lipid Solubilizers/Detergents:

### APO-10

### APO-12

### Big CHAP

### Big CHAP, Deoxy

### BRIJ® 35, PROTEIN GRADE® Detergent, 30% Solution

### BRIJ® 35, PROTEIN GRADE® Detergent, 10% Solution, Sterile-Filtered

### C12E6

### C12E8

### C12E9

### Cyclohexyl-n-ethyl-β-D-maltoside, ULTROL® Grade

### Cyclohexyl-n-hexyl-β-D-maltoside, ULTROL® Grade

### Cyclohexyl-n-methyl-β-D-maltoside, ULTROL® Grade

n-Decanoylsucrose
n-Decyl-β-D-maltopyranoside, ULTROL® Grade 252718
n-Decyl-β-D-thiomaltoside, ULTROL® Grade

### Digitonin, High Purity

### Digitonin, Alcohol-Soluble, High Purity

n-Dodecanoylsucrose 324374
n-Dodecyl-β-D-glucopyranoside 324355

### ELUGEN™ Detergent, 50% Solution

### GENAPOL® C-100, PROTEIN GRADE® Detergent, 10% Solution

### GENAPOL® X-80, PROTEIN GRADE® Detergent, 10% Solution

### GENAPOL® X-100, PROTEIN GRADE® Detergent, 10% Solution

n-Heptyl-β-D-glucopyranoside
n-Heptyl-β-D-thioglucopyranoside, ULTROL® Grade, 10% Solution
n-Hexyl-β-D-glucopyranoside

### MEGA-8, ULTROL® Grade

### MEGA-9, ULTROL® Grade

### MEGA-10, ULTROL® Grade

n-Nonyl-β-D-glucopyranoside

### NP-40, PROTEIN GRADE® Detergent, 10% Solution

n-Octanoyl-β-D-glucosylamine (NOGA)
n-Octanoylsucrose
n-Octyl-β-D-glucopyranoside
n-Octyl-β-D-glucopyranoside, ULTROL® Grade
n-Octyl-β-D-maltopyranoside
n-Octyl-β-D-thioglycopyranoside, ULTROL® Grade

### PLURONIC® F-127, PROTEIN GRADE® Detergent, 10% Solution

### TRITON® X-100

### TRITON® X-100, PROTEIN GRADE® Detergent, 10% Solution

### TRITON® X-100, Molecular Biology Grade

### TRITON® X-100, Hydrogenated

### TRITON® X-100, Hydrogenated, PROTEIN GRADE® Detergent, 10% Solution

### TRITON® X-114, PROTEIN GRADE® Detergent, 10% Solution

### TWEEN® 20

### TWEEN® 20, Molecular Biology Grade

### TWEEN® 20, PROTEIN GRADE® Detergent, 10% Solution

### TWEEN® 80, PROTEIN GRADE® Detergent, 10% Solution

n-Undecyl-β-D-maltoside, ULTROL® Grade

### Zwitterionic Lipid Solubilizers/Detergents:

### ASB-14

### ASB-16

### CHAPS

### CHAPSO

### DDMAB

### DDMAU

### EMPIGEN BBS® Detergent, 30% Solution

### Lauryldimethylamine Oxide (LDAO), 30% Solution

### ZWITTERGENT® 3-08 Detergent

### ZWITTERGENT® 3-10 Detergent

### ZWITTERGENT® 3-12 Detergent

### ZWITTERGENT® 3-14 Detergent

### ZWITTERGENT® 3-16 Detergent

Of particular interest to applicants are Triton and certain phytochemical lipid solubilizers, such as the Saponin group. The conjugation of butyric, caproic, caprylic, lauric, myristic, palmitic, stearic, arachidic, behenic, oleic, linoleic, alpha-linolenic, arachidonic, eicosapentaenoic, docosahexaenoic acid, euric acid with a Triton compound or with a Saponin will synergistically add solubilizing power to a systemic solubilizer, rendering such solubilizer capable of ubiquitously attacking atherosclerotic plaques and fatty deposits.

Applicants disclose also another way to enable the Fatty Acids conjugated with those biliary compounds which are confined/sequestered in the entero-hepatic circulation i.e. the administration of said conjugates via routes of administration which bypass the entero-hepatic circulation for bio-availability in the systemic circulation:
A) Rectal, for instance in the form of a suppository.
B) Subcutaneous via injection for prompt or slow release delivery of the substance.
C) Intramuscular for prompt or slow release of the substance in a depo form.
D) Intravenous
E) Intradermal.
F) Oral mucous membrane, such as sublingual
G) Inhalation in form of inhaled microcrystals or aerosol.
H) Others, such as vaginal or intraperitoneal route.

What we claim is:

1. A pharmacological compound aimed at ubiquitous dissolutions of lipids in a human body including lipid components in atherosclerotic plaques in the arterial circulatory system and adipose tissues in the subcutaneous layers of the skin and in other deposits of adipose tissue, comprising:
a fatty acid conjugated with
a systemic circulation biocompatible lipid solubilizer that substantially escapes absorption by the entero-hepatic circulation,
said resulting conjugated compound being capable of producing a sustained bioavailability in the systemic circulation to access and cause ubiquitous dissolution of fatty deposits including the adipose tissue deposits and the lipid components of atherosclerotic plaques.

2. The conjugated compound of claim 1 wherein the systemic biocompatible lipid solubilizer that substantially escapes absorption by the entero-hepatic circulation is hyodeoxycholic acid.

3. The pharmacological compound of claim 1 wherein the systemic biocompatible lipid solubilizer that substantially escapes absorption by the entero-hepatic circulation is a phytochemical detergent.

4. The pharmacological compound of claim 3, wherein the phytochemical detergent is a saponin.

5. The pharmacological compound of claim 1 wherein the systemic biocompatible lipid solubilizer that substantially escapes absorption by the entero-hepatic circulation is a ionic detergent.

6. The pharmacological compound of claim 1 wherein the systemic biocompatible lipid solubilizer is a non ionic detergent.

7. The pharmacological compound of claim 6, wherein the non ionic detergent acting as a systemic biocompatible lipid solubilizer is a Triton compound.

8. The pharmacological compound of claim 1 wherein the systemic biocompatible lipid solubilizer that substantially escapes absorption by the entero-hepatic circulation is a zwitterionic detergent.

9. A pharmacological compound aimed at the ubiquitous dissolutions of lipids in a human body including lipid components in atherosclerotic plaques in the arterial circulatory system and adipose tissues in the subcutaneous layers of the skin and in other deposits of adipose tissue, comprising:
a fatty acid conjugated with
a biliary salt, or biliary salt precursor or derivative,
said resulting conjugated compound being administrable to a human body via a route that bypasses the entero-hepatic circulation, said route of administration enabling the conjugated compound to be capable of producing a sustained bioavailability in the systemic circulation to access and cause ubiquitous dissolution of fatty deposits including the adipose tissue deposits and the lipid components of atherosclerotic plaques.

10. The pharmacological compound of claim 9, wherein the route of administration comprises mucous membrane.

11. The pharmacological compound of claim 9, wherein the route of administration is a transdermal route of administration.

12. The pharmacological compound of claim 9, wherein the route of administration is a subcutaneous route of administration.

13. The pharmacological compound of claim 9, wherein the route of administration is an intramuscular route of administration.

14. The pharmacological compound of claim 9, wherein the route of administration is an intravenous route of administration.

15. The pharmacological compound of claim 9, wherein the route of administration is an intradermal route of administration.
